(19) United States
(12) Plant Patent Application Publication
(10) Pub. No.: US 2002/0199224 P1
(43) Pub. Date: Dec. 26, 2002

(54) Miniature rose plant 'POULdata'

(76) Inventors: L. Pernille Olesen, Fredensborg (DK); Mogens N. Olesen, Fredensborg (DK)

Correspondence Address: Poulsen Roser Pacific, Inc., 620 S. Front Street, Central Point, OR, 97502, US

(21) Appl. No.: 10/136,453

(22) Filed: Apr. 29, 2002

Related U.S. Application Data
(63) Continuation of application No. 09/619,741, filed on Jul. 19, 2000.

(51) Int. Cl. A01H 5/00

(52) U.S. Cl. PLT/121

(57) ABSTRACT

A new miniature rose plant which has abundant, non-fading, pink flowers and attractive foliage. The variety successfully propagates from softwood cuttings and is suitable for year round production in commercial glasshouses. This new and distinct variety has shown to be uniform and stable in the resulting generations from asexual propagation.

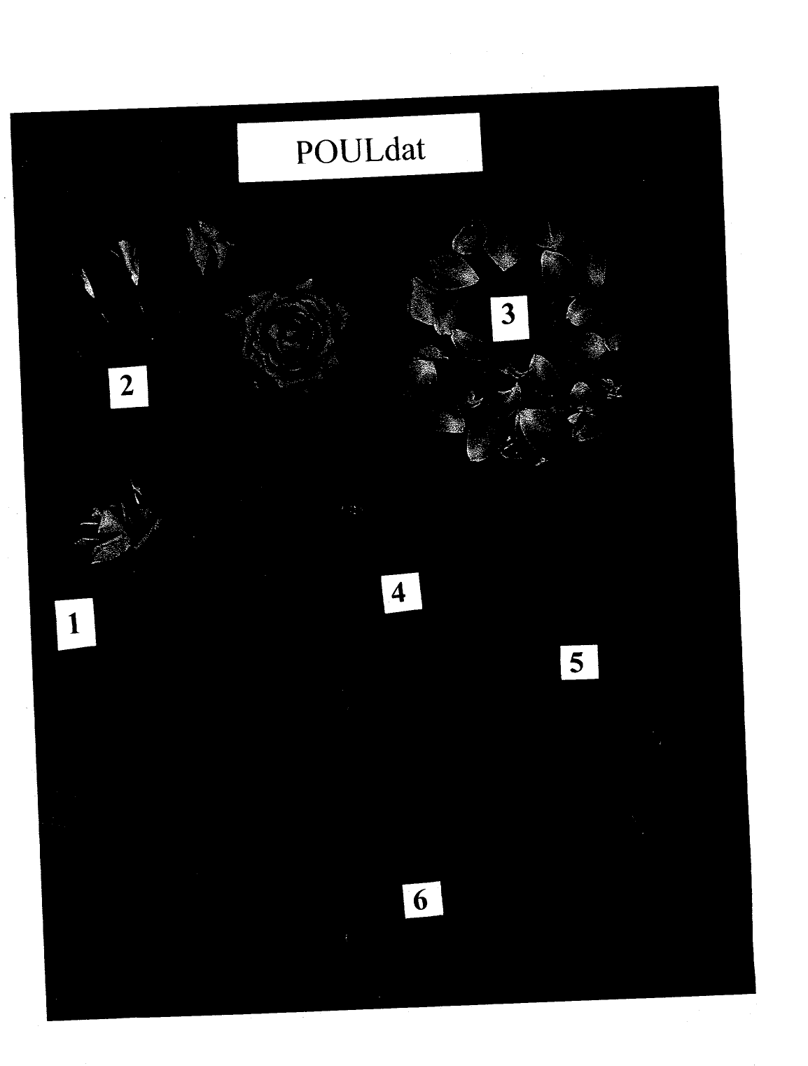

### SUMMARY OF THE INVENTION

The present invention constitutes a new and distinct variety of miniature rose plant which originated from a controlled crossing between an unnamed seedling and ‘POULhappy’. The two parents were crossed and the resulting seeds were planted in a controlled environment. The new variety is named ‘POULdata’. The new rose may be distinguished from its seed parent, an unnamed seedling, by the following combination of characteristics:

1. The unnamed seedling is a pink miniature with bigger flowers than ‘POULdata’.

The new variety may be distinguished from its pollen parent, ‘POULhappy’, a rose variety with U.S. Pat. No. Plant 9,483 issued on Mar. 26,1996, created by the same inventors, by the following combination of characteristics:

1. ‘POULhappy’'s flowers are orange-red, where those of ‘POULdata’ are pink.

2. The flowers of ‘POULhappy’ are smaller thaan those of ‘POULdata’.

3. ‘POuLhappy’ is shorter in habit than ‘POULdata’.

The objective of the hybridization of this rose variety for commercial culture was to create a new and distinct variety with unique qualities, such as:

1. Uniform and abundant flowers;

2. Vigorous and compact growth;

3. Year-round flowering under glasshouse conditions;

4. Suitability for production from softwood cuttings in pots;

5. Durable flowers and foliage which make a variety suitable for distribution in the floral industry.

The combination of qualities of this variety represents significant improvement over previously available commercial cultivars of this type and distinguishes ‘POULdata’ from all other varieties of which we are aware. As part of their rose development program, L. Pernille Olesen and Mogens N. Olesen germinated the seeds from the aforementioned hybridization and conducted evaluations on the resulting seedlings in a controlled environment in Fredensborg, Denmark.

‘POULdata’ was selected by the inventors as a single plant from the progeny of the hybridization in Fredensborg, Denmark, in Spring, 1996. Asexual reproduction of ‘POULdata’ by cuttings and traditional budding was first done by L. Pernille and Mogens N. Olesen in Fredensborg, Denmark in July of 1996. This initial and other subsequent propagations conducted in controlled environments have demonstrated that the characteristics of ‘POULdata’ are true to type and are transmitted from one generation to the next.

### BRIEF DESCRIPTION OF THE DRAWING

The accompanying color illustration shows as true as is reasonably possible to obtain in color photographs of this type, the typical characteristics of the buds, flowers, leaves, and stems of ‘POULdata’. Specifically illustrated in SHEET 1:

1. Stem showing branching and the attachment of leaves, buds, and peduncles;

2. Flower bud, partially opened bud, and open bloom;

3. Flower petals, detached;

4. Sepals, receptacle, and pedicel;

5. Flowering stem as well as a bare stem exhibiting thorns;

6. Leaves.

### DETAILED DESCRIPTION OF THE VARIETY

The following is a description of ‘POULdata’, as observed in its growth in glasshouses in Half Moon Bay, California, on plants aged sixteen weeks. Color references are made using The Royal Horticultural Society (London, England) Colour Chart, 1995, except where common terms of color are used. For a comparison, several physical characteristics of the rose variety ‘POULeden’, a miniature rose variety from the same inventors described and illustrated in U.S. Plant patent application Ser. No. 09/113,420 dated Jul., 10,1998 are compared to ‘POULdata’ in Chart 1.

CHART 1

| | ’POULdata’ | ’POULeden’ |
|---|---|---|
| Petal Count | Double: 35 to 40 | Double: 30 to 35 |
| Plant Height | 20 to 25 cm | 16 to 18 cm |
| Basal Petal Spots | Outer Petals: Green-White | Outer Petals: |
| | Group 157C. | Yellow Group 3A. |
| | Inner Petals: White Group | Inner Petals: |
| | 155A | Yellow Group 9B. |

Parents: Unnamed Seedling×POULhappy

Classification:

Botanical.—Rosa hybrida.

Commercial.—Miniature.

### FLOWER AND FLOWER BUD

Blooming habit: Continuous.

Flower bud:

Size.—Upon opening, 22 to 25 mm in length from base of receptacle to end of bud.

Bud form.—Pointed ovoid.

Bud color.—As sepals unfold, Red Group 43D. Red Group 43D at ¼ opening.

Sepals.—Average length is 18 mm; average width is Green Group 137C. with intonations of Green Group 143C. Weak foliaceous appendages on two to three of the five sepals. Surfaces of sepals moderately pubescent. Stipitate glands are present on sepals and peduncle.

Receptacle.—Surface: Smooth. Shape: Urceolate Size: 5 to 6 mm (h)×6 to 8 mm (w). Color: Yellow Green Group 144B.

Peduncle.—Surface: Small amount of minute thorns, many stipitate glands. Length: Length variable, from 28 to 54 mm average length. Color: Yellow Green Group 144B. Strength: Upright.

Borne.—Generally with 3 to 4 buds per flowering stem.

Flower bloom:

Fragrance.—None.

Duration.—As a pot plant, flowers last from 11 to 14 days. As a cut flower 6 to 8 days.

Size.—Small for a 15 cm pot rose. Average flower diameter is 45 mm when open.

Form.—Shape of flower when viewed from the side: Upon opening, upper part: Flat. Upon opening, lower part: Flattened convex. Open flower, upper part: Flattened convex. Open flower, lower part: Flattened convex.

Petalage.—Double. Average range:35 to 40 petals under normal conditions with 3 to 4 petaloids.

Color:

Upon opening petals.—Outermost petals: Upper Surface: Red Group 49A. Reverse Side: Red Group 49A. Innermost petals: Upper Surface: Red Group 49A. Reverse Side: Red Group 49B.

Upon opening, basal petal spots.—Outermost petals: Outer Side: Green-White Group 157D. Inner Side: Green-White Group 157C. Innermost petals: Outer Side: White Group 155A. Inner Side: White Group 155A.

After opening, petals.—Outermost petals: Upper Surface: Red Group 50C. Reverse Side: Red Group 54D. Innermost petals: Upper Surface: Red Group 50C. Reverse Side: Red Group 54C.

After Opening, basal petal spots.—Outermost petals: Outer Side: White Group 155C. Inner Side: White Group 155C. Innermost petals: Outer Side: White Group 155C. Inner Side: White Group 155C.

General Tonality: On open flower, Red Group 49A. No change in the general tonality at the end of the 4^{th} day. Afterwards, general tonality is Red Group 56A.

Petals:

Petal Reflex.—Outermost petals reflex strongly; the rest only moderately.

Petal Margin.—Uniform.

Shape.—Deltoid.

Petaloids.—Present: 3 to 4.

Thickness.—Average.

Arrangement.—Informal.

Reproductive Organs:

Pollen.—Color: Yellow-Orange Group 21B. Quantity: Scant.

Anthers.—Size: 2 mm long. Color: Yellow Group 8C.

Filaments.—Color: Yellow-Green Group 154C. Length: 4 to 6 mm. Quantity: 35 to 40.

Stigmas.—Even or very slightly superior in location to anthers. Color: Greyed-Yellow Group 160D.

Styles.—Color: Greyed-Yellow Group 1C. Length: 4 to 7 mm. Quantity: 35 to 40.

### PLANT

Plant growth: Vigorous, upright to bushy. When grown as a 12 cm pot plant, the average height of the plant itself is 25 cm to 30 cm and the average width is 30 cm to 33 cm.

Stems:

Color.—Young wood: Yellow-Green Group 144A. Older wood: Yellow-Green Group 144A.

Thorns.—Incidence: Moderate. Size: Average length: 3 to 4 mm. Color: Greyed-Yellow Group 160C. Shape: Linear.

Surface.—Young wood: Smooth. Older wood: Smooth.

Length.—Under typical greenhouse production conditions, stem length is 25 to 30 cm. Internode length varies from 8 to 10 cm.

Plant foliage: Number of leaflets on leaves in middle of the stem: 5 leaflets:

Leaf size.—21 to 28 mm (1)×13 to 18 mm (w).

Abundance.—Average abundance.

Color.—Upper Leaf Surface: Yellow-Green Group 147A. Lower Leaf Surface: Green Group 137C. Juvenile foliage: Yellow-Green Group 144A with intonations of Yellow-Green Group 144C.

Anthocyanin intonation.—Location: Margins on edges of young leaves. Color: Red Group 46A.

Plant leaves and leaflets:

Stipules.—Size: 5 to 8 mm in length. Color: Center is Yellow-Green Group 144C and edges are Green Group 141A. Presence of stipitate glands: Limited.

Petiole.—Length: 3 mm to 6 mm. Color: Center is Yellow-Green Group 145B, margins are Yellow-Green Group 146B. Underneath: Yellow-Green Group 144A. No thorns noted. Margins: Green Group 146B.

Rachis.—Color: Yellow-Green Group 144C. Underneath: Yellow-Green Group 144A. No thorns noted. Margins: Green Group 138A.

Leaflet.—Edge: Finely Serrated. Shape: Pointed oval. Texture: Leaf is thin and slightly glossy.

Disease resistance: Above average resistance to mildew, black spot, and Botrytis under normal growing conditions in Half Moon Bay, Calif.

Cold hardiness: ‘POULdata’ has been found to be resistant to damage from cold, heat and drought damage in USDA Zone 7.

1. A new and distinct variety of rose plant of the miniature class, substantially as herein illustrated and described as a distinct and novel rose variety due to its abundant, pink flowers, vigorous and compact growth, year round flowering under glasshouse conditions, suitability for production from softwood cuttings in pots, and durable flowers and foliage which make the variety suitable for distribution in the floral industry:
